(19) United States
(12) Patent Application Publication
(10) Pub. No.: US 2005/0096608 A1
(43) Pub. Date: May 5, 2005

(54) Two-component aspiration cannula

(76) Inventors: Werner Mannschedel, Langenau (DE); Barbara Muller, Langenau (DE)

Correspondence Address: FROMMER LAWRENCE & HAUG, 745 FIFTH AVENUE- 10TH FL., NEW YORK, NY, 10151, US

(21) Appl. No.: 10/961,874

(22) Filed: Oct. 8, 2004

(30) Foreign Application Priority Data
Oct. 10, 2003 (DE) DE 103 47 829.9

(51) Int. Cl. A61M 25/00

(52) U.S. Cl. 604/264; 433/91

(57) ABSTRACT

The present invention relates to an aspiration cannula for the aspiration of fluid substances, especially saliva and blood, during dento-medical treatment.

The present invention relates, in accordance with its generic type, to an aspiration cannula for aspirating fluid substances, especially saliva and blood, during dento-medical treatment. It relates especially to an aspiration cannula which comprises an elongate tubular element having an internal fluid-guiding passage, one end of the fluid-guiding passage opening into a connection orifice for connection to an aspiration device and the other end opening into an aspiration orifice for aspirating fluids. In use, the aspiration orifice of the aspiration cannula is intended for introduction into the oral cavity of a patient.

Aspiration cannulas of such a kind are nowadays used routinely and in large numbers during dento-medical treatment. In manufacture, the aspiration cannulas are generally produced from a hard, non-resilient plastics material by injection moulding.

However, it has been found in practical use that the hard, non-resilient plastics material of the aspiration cannulas is very unpleasant for the patient when it comes into contact with the sensitive anatomical structures of the oral cavity and can even cause pain. In addition, it has been found that aspiration cannulas of such a kind are subject to the production of a great deal of noise during the aspiration process, that aspiration noise being transmitted in extremely unpleasant manner to the auditory system and further anatomical structures of the patient when the aspiration cannula comes into contact with an anatomical structure of the oral cavity.

For the user, that is to say the dentist or frequently the dental assistants, manually gripping and holding tight the hard, smooth-surface aspiration cannulas is, in the long run, tiring and onerous. That is especially the case when the user is wearing protective gloves, which are usually made from latex, in order to avoid the transmission of transmissible infectious diseases, for example HIV or hepatitis.

Because the aspiration cannulas made from hard plastics material are generally produced by injection moulding, the aspiration cannulas have at least one mould gate mark; in industrial mass-production, that gate mark can, however, be deburred only inadequately because of the hard material and consequently almost always has a sharp-edged rim. Although it is customary nowadays for the dentist and dental assistants to wear protective gloves when treating patients in order to avoid transmissible infections, it sometimes happens that latex gloves are torn by the sharp burr of a gate mark, in which case infection of the patient or dentist/dental assistants may occur in undesirable manner. When a sharp-edged gate mark of such a kind is located in that region of the aspiration cannula which is introduced into the oral cavity, that is to say especially close to or at the aspiration end of the aspiration cannula, the patient can even be injured by the sharp burr of the gate mark when the aspiration cannula is moved. When the aspiration cannula is moved, for example, along the oral mucosa, the oral mucosa can, for example, be cut by the sharp burr of the gate mark.

If an aspiration cannula produced by injection moulding has a wing-like widening (“pad”) at its aspiration end, that widening always has, for production reasons, a centrally arranged, sharp-edged burr, which can, like the sharp-edged gate mark, injure the patient and/or damage the user's protective glove.

Furthermore, it has been found to be disadvantageous from an economic and ecological point of view that the aspiration cannulas are produced solely in the form of disposable articles, which is to say that the aspiration cannulas are thrown away after being used once because use in the intended manner results in them coming into contact with potentially infectious fluids such as saliva or blood. For a dentist's practice this gives rise to the not inconsiderable cost of purchasing the cannulas because of the large number of patients treated. Disposing of the potentially infectious material is also costly.

The problem of the present invention is to avoid the afore-mentioned disadvantages of the aspiration cannulas known from the prior art.

To solve the problem, the present invention proposes an aspiration cannula for the aspiration of fluid substances, especially saliva and blood, during dento-medical treatment, which comprises an elongate tubular element having an internal fluid-guiding passage, one end of which fluid-guiding passage opens into a connection orifice for connection to an aspiration device and the other end of which fluid-guiding passage opens into an aspiration orifice for aspirating fluids, a characteristic feature of the invention being that the tubular element is surrounded at least in part by a resiliently deformable material and is of integral construction therewith.

In other words, the elongate tubular element of the aspiration cannula is surrounded at least in part by a soft material, it being possible for the tubular element to be made from a non-resilient hard material. In addition, the aspiration cannula is advantageously of integral construction, that is to say the soft, resiliently deformable material surrounding the tubular element is joined to the tubular element, forming a monolithic structure.

When the aspiration cannula is produced entirely by injection moulding, that is to say when the resiliently deformable material surrounding the tubular element is also produced by injection moulding, the gate mark of the soft, resiliently deformable material is without a remaining sharp-edged burr even in industrial mass-production. As an alternative thereto, it is possible, when only the tubular element is produced by injection moulding, for a gate mark having a sharp-edged burr to be covered over by the soft, resiliently deformable material surrounding at least in part the tubular element.

When the tubular element has a gripping region intended for manually gripping the aspiration cannula, it is especially advantageous for the gripping region to be surrounded by the resiliently deformable material, the soft, resiliently deformable material making it possible for the aspiration cannula to be securely gripped and held tight by the user. In the long run, therefore, the user tires less than in the case of the conventional, hard aspiration cannulas. This also means that the user can grip the aspiration cannula without the risk of damage to his protective glove, which greatly facilitates handling of the aspiration cannula. For the patient there is, in addition, no risk of injury to anatomical structures of the oral cavity from a gate mark with a sharp-edged burr located in the gripping region.

Advantageously, the gripping region can also be provided with a surface structure, for example grooving.

It is also especially advantageous when, as a result of the resiliently deformable material in the gripping region of the aspiration cannula, haptic recognition of the aspiration-orifice end of the aspiration cannula is possible. By that means, the user can ascertain, by gripping the aspiration cannula “blind”, whether the aspiration cannula is correctly located in his hand for use in the aspiration of fluids. There is no need for a visual check (which requires the user to avert his gaze from the treatment site), which advantageously facilitates use of the aspiration cannula in practice. The gripping region can, for that purpose, be constructed, for example, in a conical shape, e.g. so that a conically tapering shape indicates the aspiration orifice.

When the aspiration-orifice end of the tubular element is in the form of a wing-like widening (pad) for keeping the aspiration orifice spaced away from anatomical structures of the oral cavity of the patient, as is known from the prior art, it has been found to be especially advantageous for that pad to be surrounded by the soft resilient material. As a result there is created a soft contact region of the aspiration cannula for contact with the patient, which a patient will find substantially more pleasant than in the case of the hard aspiration cannulas of the prior art. In addition, covering the pad with the soft resiliently deformable material advantageously gives rise to production of less noise during aspiration, and transmission of the aspiration noise into the hearing and other anatomical structures of the patient is reduced. For the patient, this means a substantial improvement in comfort during dento-medical treatment.

When the soft, resiliently deformable material surrounding the pad is produced by injection moulding, the gate mark can soft, no sharp-edged gate mark or sharp-edged central burr is formed. As an alternative or additionally, gate marks having sharp-edged rims or centrally arranged sharp-edged burrs caused by production can be covered over by the soft, resiliently deformable material, thereby avoiding potential injury to the patient as a result of sharp-edged rims in that region of the aspiration cannula which is intended for patient contact.

From an economical point of view, preference is given, in accordance with the invention, to the aspiration cannula being sterilisable, especially autoclavable, which makes it possible for the aspiration cannula to be used for different patients, as a result of which purchase and disposal costs are reduced.

Preference is furthermore given, in accordance with the invention, to the resiliently deformable material being provided with a coding, especially a colour coding. By that means it is possible, for example, for manufacturer, size and characteristic details to be marked on the aspiration cannula so that it is not necessary, for example, for different aspiration cannulas to be stored separately from one another because the aspiration cannulas can be readily distinguished from one another. Furthermore, confusion of one aspiration cannula with another is prevented.

In accordance with the invention preference is given to the tubular element comprising a material which can be shaped by injection moulding, in which case it may be, for example, a thermoplastic material, especially polypropylene, polystyrene, PE, PVC, PMNA or ABS, or combinations thereof.

Furthermore, preference is given, in accordance with the invention, to the soft, resiliently deformable material comprising rubber or a rubber-like material such as, for example, EPDM, SBR, NR, butyl rubber, natural rubber, latex, polychloroprene. Special preference is given to its being a thermoplastic elastomer, especially a block copolymer or an elastomer alloy or polymer blend.

Elastomer alloys and polymer blends have thermoplastic and elastomeric contents which, as a result of mixing different components, result in new materials having new properties. A large number thereof have been produced hitherto. The most widespread—and especially preferred according to the invention—is the group of EPDM/PP blends, NR/PP blends (thermoplastic natural rubber), NBR/PP blends, IIR/PP blends, EVA/PVDC blends and NBR/PVC blends.

Furthermore, in accordance with the invention, the rubber-like material of the resiliently deformable material surrounding the tubular element can be silicone or a silicone compound.

Especially advantageously, the aspiration cannula according to the invention can be produced in monolithic form by injection moulding, especially 2-component injection moulding, or extrusion. It is likewise possible, for example, for solely the tubular element of the aspiration cannula to be produced by injection moulding or extrusion and for the resiliently deformable material surrounding at least in part the tubular element to be subsequently applied to the tubular element by means of a dipping method, wherein the tubular element is dipped into a dipping bath comprising resiliently deformable material. As an alternative thereto, it is possible for the resiliently deformable material surrounding the tubular element to be applied to the tubular element in another manner known from the prior art and joined in a force-based and/or shape-based connection to the tubular element, especially bonded thereto.

The invention will be explained below in further detail by means of an exemplary embodiment referring to the accompanying drawing.

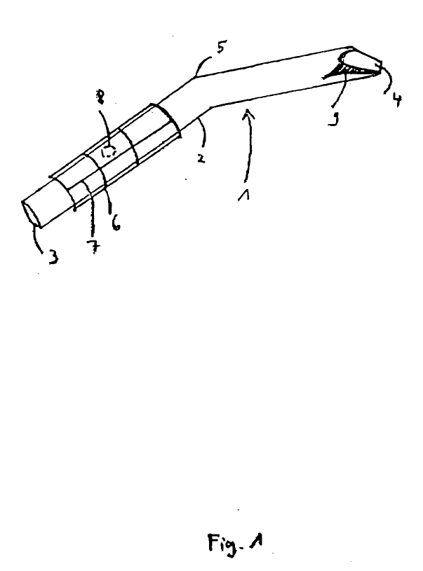

FIG. 1 shows an embodiment of the aspiration cannula according to the invention.

As can be seen from FIG. 1, an aspiration cannula 1 according to the invention comprises a tubular element 2. The tubular element 2, which is made from polypropylene, has an internal fluid-guiding passage, one end of which opens into a connection orifice 3 for connection to an aspiration device (not shown) and the other end of which opens into an aspiration orifice 4 for aspirating fluids. The aspiration device 1 has a bend 5 for easier handling in practical use. A gripping region of the aspiration cannula is surrounded by a soft, resiliently deformable material 6. The soft, resiliently deformable material is a thermoplastic elastomer (SEBS). The resiliently deformable material has, for better handling of the aspiration cannula, grooving 7, which consists of longitudinal and transverse grooves. As a result of the resiliently deformable material 6, a gate mark 8 (shown in diagrammatic form by a broken line) having a sharp-edged rim on the tubular element 2 is covered over. The aspiration cannula 1 is provided with a wing-like widening 9 (pad) of the aspiration-orifice end of the aspiration cannula.

The aspiration cannula of integral construction, which is shown in FIG. 1, was produced by a 2-component injection moulding method.

1. Aspiration cannula for the aspiration of fluid substances, especially saliva and blood, during dento-medical treatment, which comprises an elongate tubular element having an internal fluid-guiding passage, one end of which fluid-guiding passage opens into a connection orifice for connection to an aspiration device and the other end of which fluid-guiding passage opens into an aspiration orifice for aspirating fluids, wherein the tubular element is surrounded at least in part by a resiliently deformable material and is of integral construction therewith.

2. Aspiration cannula according to claim 1, wherein the tubular element is made from a non-resilient material.

3. Aspiration cannula according to claim 1, wherein the tubular element has a gripping region intended for manually gripping the aspiration cannula, which gripping region is surrounded by the resiliently deformable material.

4. Aspiration cannula according to claim 3, wherein the gripping region surrounded by the resiliently deformable material makes possible haptic recognition of the aspiration-orifice end of the aspiration cannula.

5. Aspiration cannula according to claim 4, wherein the gripping region surrounded by the resiliently deformable material has a conical shape.

6. Aspiration cannula according to claim 3, wherein the gripping region surrounded by the resiliently deformable material has a surface structure, especially grooving.

7. Aspiration cannula according to claim 1, wherein the aspiration-orifice end is surrounded by the resiliently deformable material.

8. Aspiration cannula according to claim 1, wherein the aspiration-orifice end of the tubular element is in the form of a pad for keeping the aspiration orifice spaced away from anatomical structures of the oral cavity of the patient, which pad is surrounded by the resiliently deformable material.

9. Aspiration cannula according to claim 1, wherein the tubular element is produced by injection moulding and has at least one gate mark, the at least one gate mark being covered over by the resiliently deformable material.

10. Aspiration cannula according to claim 1, wherein the resiliently deformable material is provided with a coding, especially colour coding.

11. Aspiration cannula according to claim 1, which is sterilisable, especially autoclavable.

12. Aspiration cannula according to claim 1, wherein the tubular element consists of a material which can be shaped by injection moulding.

13. Aspiration cannula according to claim 12, wherein the tubular element comprises thermoplastic materials, especially polypropylenes, polystyrenes, ABS and combinations thereof.

14. Aspiration cannula according to claim 1, wherein the resiliently deformable material is rubber or a rubber-like material.

15. Aspiration cannula according to claim 14, wherein the rubber-like material is a thermoplastic elastomer, especially a block copolymer or an elastomer alloy.

16. Aspiration cannula according to claim 14, wherein the rubber-like material is silicone or a silicone compound.

17. Aspiration cannula according to claim 1, produced by injection moulding or extrusion.

18. Aspiration cannula according to claim 1, wherein the resiliently deformable material surrounding at least in part the tubular element is applied by a dipping method.

19. Aspiration cannula according to claim 1, wherein the resiliently deformable material surrounding at least in part the tubular element is joined in a force-based and/or shaped-based connection to the tubular element, especially bonded thereto.
